(19) United States
(12) Patent Application Publication
(10) Pub. No.: US 2004/0220388 A1
(43) Pub. Date: Nov. 4, 2004

(54) Novel heterodimeric fusion proteins

(76) Inventors: Nico Mertens, Melsele (BE); Johan Grooten, Lovendegem (BE)

Correspondence Address: MUSERLIAN AND LUCAS AND MERCANTI, LLP, 475 PARK AVENUE SOUTH, NEW YORK, NY, 10016, US

(21) Appl. No.: 10/312,923

(22) PCT Filed: Jun. 29, 2001
(86) PCT No.: PCT/EP01/07557

(30) Foreign Application Priority Data
Jun. 30, 2000 (EP) 00202306.7

(51) Int. Cl. C07K 16/30; A61K 39/395

(52) U.S. Cl. 530/388.8

(57) ABSTRACT

The present invention relates to the production of bispecific or multispecific, bi- or tetravalent antibodies using recombinant DNA methods and recombinant production methods. The resulting antibody consists of one or two diabody molecules that are heterodimerized by creating a fusion protein with the CL and CH1 immunoglobulin constant domains.

### FIELD OF THE INVENTION

The invention relates to the production of bispecific or multispecific, bi- or tetravalent antibodies using recombinant DNA methods and recombinant production methods. The resulting antibody consists of one or two diabody molecules that are heterodimerized by creating a fusion protein with the CL and CH1 immunoglobulin constant domains.

### BACKGROUND OF THE INVENTION

Bispecific antibodies are antibodies that can bind with at least two different antigens. By their nature, bispecific antibodies have potential use in the preparation of both therapeutic and diagnostic reagents. Especially in therapeutic settings, bispecific antibodies can have an improved effect over monospecific antibodies. Careful choice of the target specificities will enable the user to create an effect beyond the use of monospecific antibodies. Mono- or multivalent bispecific antibodies or multivalent antibodies can have an improved activity over natural antibodies when used as a diagnostic agent in vitro as well as in vivo. Bispecific antibodies can be created in different ways and forms.

Bispecific IgG (BsIgG) molecules can be created by chemical reassociation of monovalent L and H fragments (Brennan et al., 1985), by hybrid hybridoma (Milstein and Cuello, 1983) (U.S. Pat. No. 4,474,893, U.S. Pat. No. 4,714,681), or by engineering knobs-into-holes complementarity into both H-chains (Ridgway et al., 1996) (WO9850431). Tetravalent bispecific antibodies can be created by chemical crosslinking of two monoclonal antibodies (Bs(IgG)2) (Karpovsky et al., 1984) (U.S. Pat. No. 4,676,980).

Using F(ab)′ fragments as building blocks, multivalent bispecific antibodies can also be created by chemical crosslinking of two or more Fab′ molecules (Bs(Fab′)₂) (Glennie et al., 1987) (WO9103493, WO9804592). A genetically controlled heterodimerization of a Bs(Fab′)₂ molecule was described by Kostelny et al., 1992, where the F(ab′)-molecules were fused to a fos and a jun heterodimerization domain (U.S. Pat. No. 5,932,448).

The smallest functional binding unit of an antibody constitutes of the variable domains of both the heavy (VH) chain and the light (VL) chain. However, VH and VL do not interact in a stable way with each other. Different solutions have been proposed to stabilize these domains. The introduction of a disulfide bond between the domains was proposed, but usually led to loss of affinity and requires protein engineering on each particular domain pair. Another solution was to connect both domains with a flexible linker, long enough to bridge the distance between the C-terminus of one domain with the N-terminus of the other domain. This class of molecules is referred to as single chain variable domains (scFv) (U.S. Pat. No. 4,946,778, U.S. Pat. No. 5,091,513). Through this linker, the domains could still disengage, but stay connected and will have a high chance of re-engaging with each other. This phenomenon is often referred to as “breading” of the molecule. The scFv approach was more universal and was widely adapted, but led to the notion that scfv were not very stable molecules, probably due to the “breathing” of the domains and the vulnerability of the non-structured peptide linker domain to proteases present in body fluids and tissue. Also, some scFv molecules have been shown to be unstable in respect to long-term storage and repeated freeze-thawing procedures.

Bispecific antibodies comprising scFv molecules (U.S. Pat. No. 5,091,513) can be constructed by chemical coupling of 2 scFv molecules (Kipriyanov et al., 1994) (U.S. Pat. No. 5,534,254), or by creating mini-antibodies by coupling the scFv molecules to a small heterodimerizing helix (Pack and Pluckthun, 1992) (U.S. Pat. No. 5,910,573), by coupling the scFv molecules to an Fc tail (Hayden et al., 1994), or by genetic coupling of both scFv molecules through a polypeptide linker (Mack et al., 1995) (U.S. Pat. No. 5,637,481). When this linker contains a heterodimerizing helix, a tetravalent Bs(scFv)₂)₂ (BiDi-body) is formed (Muller et al., 1998a). The scFv molecules can also be coupled N-terminally to immunoglobulin constant domains such as CH3 (Hu et al., 1996) (WO9409817) or CL (McGregor et al., 1994) to increase their molecular weight, or to both CL and CH1 (Muller et al., 1998b) (WO0006605) to also improve upon heterodimerization. ScFv molecules have also been coupled C-terminally to either the CH3 domain of a full-length IgG, or to the hinge region of a F(ab′)₂ (Coloma and Morrison, 1997) (WO9509917). Efficient heterodimerization of two molecules such as scFv molecules in mammalian cells can be achieved by using the Fab-chains (L and Fd) as a heterodimerization scaffold (Schoonjans et al., 2000) (WO9937791), since this heterodimerization is controlled by a cellular quality control system involving the chaperone BiP (Lee et al., 1999). The role of BiP is accepted as a mediator or chaperone to ensure the proper formation of the CL:CH1 heterodimer.

Diabodies are dimers of two scFv molecules that cannot fold properly into one scFv molecule. Diabodies are build like scFv molecules, but usually have a short (less than 10, preferably 1-5 amino acids) peptide linker connecting both V-domains, whereby both domains can not interact intramolecular, and are forced to interact intermolecular (Holliger et al., 1993) (U.S. Pat. No. 5,837,242). A diabody thus may consist of a VH-VL chain that interacts with a similar VH-VL chain to form a dimer of the formula VH-VL:VH-VL. The term diabody chain refers to one polypeptide chain comprising one VH-VL (or VL-VH) domain sequence. The diabody chain dimers bind the antigen specified by VH and VL bivalent. Winter described the construction of bispecific diabodies by coupling the VH domain of a chosen antibody A to the VL domain of a chosen antibody B, using a peptide linker sufficiently short to inhibit the interaction of VH(A) with VL(B). Also the reverse molecule VH(B)-VL(A) is made the same way (Holliger, Griffiths, Hoogenboom, Malmqvist, Marks, McGuinness, Pope, Prospero and Winter: “Multivalent and multispecific binding proteins, their manufacture and use”, U.S. Pat. No. 5,837,242, 1998).

Bispecific diabodies are potential useful compounds in diagnosis or therapy. In order to produce a bispecific diabody, one needs to co-produce two chains that need to heterodimerize in order to form the wanted molecule, VH(A)-VL(B):VH(B)-VL(A). Since most VH domains can pair with any given VL, also the homodimers VH(A)-VL(B):VH(A)-VL(B) and VH(B)-VL(A):VH(B)-VL(A) will be formed. These by-products have to be removed in order to obtain a pure compound. Specific protein engineering techniques have been proposed to preferentially obtain the heterodimerized molecule (U.S. Pat. No. 5,807,706). Bispecific diabodies can be produced, and heterodimerization can be enhanced by engineering complementarity into the domains by protein engineering (Zhu et al., 1997) (WO9850431). This “knobs-into-holes” mutagenesis technique is however very dependent on the specific protein interface to be engineered, and can not be used to heterodimerize a given diabody pair without extensive research on stability and possible loss of binding affinity of the antibody fragments. Furthermore, possible antigenic or immunogenic alterations are introduced into the molecule. Bispecificity can also be improved by creating a single chain diabody (scDb) (Kipriyanov et al., 1999) (WO9957150). These scDb molecules can be dimerized by coupling to a CH3 domain or an Fc-fragment (Alt et al., 1999) to create multivalent binding molecules with an increased molecular weight

Apart from the problem of controlling the heterodimerization of bispecific diabodies, diabodies have a particular disadvantage for most therapeutic applications in vivo. Due to their small size, diabodies are rapidly cleared from the body by the kidney. Their short persistence time reduces their therapeutic index considerably, and increases the costs involved with application of the product. An increase in molecular weight size will increase the serum permanence and product efficacy. (Wu, A. M., Chen, W., Raubitschek, A., Williams, L. E., Neumaier, M., Fischer, R., Hu, S. Z., Odom-Maryon, T., Wong, J. Y. and Shively, J. E.: Tumor localization of anti-CEA single-chain Fvs: improved targeting by non-covalent dimers. Immunotechnology 2 (1996) 21-36).

CL:CH1 domains have been suggested as fusion partners to scFv molecules in order to create bispecific antibodies, either in bacteria (Muller et al, 1998) or in mammalian cells (WO0006605). Muller, Arndt, Strittmafter and Pluckthun (“The first constant domain (CH1 and CL) of an antibody used as heterodimerization domain for bispecific miniantibodies” FEBS Left 422 (1998) 259-64) describes the use of CL:CH1 interaction to drive heterodimerization of scFv molecules. The resulting molecule of the formula scFv-CL:scFv-CH1 was expressed in Escheichia coli. These scFv molecules are capable of folding independently and are separated from the constant domains by a sufficiently long peptide spacer region. It has been shown that in mammalian cells the CH1 domain is prevented from folding by the chaperone protein BiP in the endoplasmatic reticulum (Lee et al, 1999), until pairing with a correct CL domain. These authors and Schoonjans and Mertens (1999), WO9937791 also show evidence that the CL:CH1 interactions is not sufficient to replace BiP with CL and let the complex proceed along the secretion pathway: also the VL and VH domains need to be intact so that the complete VLCL chain can pair with the VHCH1 chain. They speculate that the variable domains need to contribute to the displacement energy to reverse the interaction with a quality control chaperone in the endoplasmic reticulum of the mammalian cell. (Schoonjans, R., Willems, A., Schoonooghe, S., Fiers, W., Grooten, J. and Mertens, N.: Fab chains as an efficient heterodimerization scaffold for the production of recombinant bispecific and trispecific antibody derivatives. J Immunol 165 (2000) 7050-7; “Multipurpose antibody derivatives” WO9937791). A similar result was obtained by Lee, Brewer, Hellman, and Hendershot: “BiP and immunoglobulin light chain cooperate to control the folding of heavy chain and ensure the fidelity of immunoglobulin assembly” Mol Biol Cell 10 (1999; 2209-19). Here, either a light chain comprising a VL chain that was incapable of folding, or an isolated CL domain could not lead to secretion of the heavy chain or the Fd fragment of the heavy chain.

Kufer, Zettl, Dreier, Baeuerle, and Borschert claim the synthesis of a scFv-CL:scFv-CH1 heterodimer in a mammalian host (Heterominibodies, WO0006605). Also Zuo et al, (2000) describe the use of CL and CH1 domains to drive heterodimerization of scFv molecules in mammalian cells. (Zuo, Jimenez, Witte and Zhu: An efficient route to the production of an IgG-like bispecific antibody. Protein Eng 13 (2000) 361-7).

Although these documents disclose the use of the CL and CH1 constant domains to obtain a heterodimer, they clearly refer to a model where the domains coupled to the CL and CH1 domains lack intrinsic affinity to one another, and are linked to each other via the interaction of the said constant domains.

By their increased interaction, diabodies are believed to be more stable antibody fragments than-scFv. Bispecific diabodies however contain non-productive side products by homodimerizing diabody chains. Furthermore, the small size (<60 kDa) of a diabody results in a rapid clearance when used in vivo. The effective time frame can then be to small to be effective. Molecules with a higher molecular weight are more preserved from this clearance in the kidneys.

The present invention is based on the unexpected and surprising finding that, when using CL and CH1 domains that are clearly dependent on extension with VL and VH domains for secretion, other fusion partners with intrinsic affinity for one another could substitute for the VL and VH domains. It was particular surprising to find that a complex and artificial molecule such as a diabody can substitute for the correctly positioned VL and VH domains, while it is predicted that the VL and VH domains incorporated in the diabody are not positioned in the same conformation or even orientation as the variable domains in a Fab molecule.

The present invention thus also improves the ratio of heterodimer formation over homodimer formation of two diabody chains. Indeed, the present invention relates to an improved method to produce heterodimeric fusion proteins by creating a heterodimeric fusion protein of the diabody chains to be heterodimerized and either the CL or the CH1 domain. After CL:CH1 association, a heterodimeric fusion protein that can comprise several fused protein domains is formed. In the molecule described by the present invention, all said fused protein domains still have intrinsic affinity to corresponding domains of the other chain in the heterodimer.

The present invention more specifically provides a method for controlled heterodimerization of one or more diabody chains, after which one or more bispecific diabodies are formed as part of one fusion protein. The term ‘controlled’ refers to the ability to determine all the specificities and the number of antigen binding sites within the fusion molecule by design.

The method of the present invention describes the use of a proteinacious heterodimerization signal for one or more diabody chains. In particular, the invention relates to a fusion protein comprising two chains, where each chain comprises one or more diabody chains and a CL or a CH1 domain. Moreover, the CL and CH1 domains are protein domains naturally found in serum, so no antigenicity is expected. Furthermore they can be disulfide stabilised, improving the stability of the final product.

### SUMMARY OF THE INVENTION

The present invention uses the heterotypic interaction of the CH1:CL domains to enhance the formation of bispecific diabodies. A diabody consists of two chains that interact with each other to constitute two antigen-binding sites. In order to produce efficiently bispecific diabodies, the heterodimerization of two different chains needs to be preferred over the homodimerization of two equal chains.

One preferred embodiment of the present invention is a novel heterodimer, where each of the two chains contain a fusion protein that consists of one or more diabody chains that are coupled to the CL or the CH1 constant immunoglobulin domain. The novel fusion chain can be of the formula VH(A)-VL(B)-CL:VH(B)-VL(A)-CH1, where the diabody chains are fused to the N-terminus of the constant domains. The novel fusion protein can also contain the diabody chains fused at the C-terminus of the constant domains and thus be of the formula CL-VH(C)-VL(D):CH1-VH(D)-VL(C). Also, but not limiting, the fusion chain can contain two diabody chains and be of the formula VH(A)-VL(B)-CL-VH(C)-VL(D):VH(B)-VL(A)-CH1-VH(D)-VL(C). In the examples mentioned, it is preferred that the VH-VL:VH-VL dimerization will constitute a functional diabody. The order of VH-VL can be reversed to VL-VH if also the order in the complementary chain is reversed.

The invention further relates to methods for making these novel heterodimers, to DNA comprising genes encoding these novel fusion proteins, to transformed host cells containing said DNA, and to the use of these novel fusion proteins for diagnostic, therapeutic or other purposes.

### BRIEF DESCRIPTION OF FIGURES

FIG. 1: schematic representation of a diabody structure fused to (A) the N-terminal part of the CL and CH1 domains, (B) to the C-terminal part of these domains when these domains are incorporated in a Fab fragment, and (C) when a diabody is fused to both the N-terminal and C-terminal part of the CL and CH1 domains. Each panel shows a representation of both an organizational scheme and a prediction of the structure of the heterodimeric fusion protein. Domains fused to CL-domain and the CL domain are coloured dark, domains fused to the CH1 domain and the CH1 domain are coloured light.

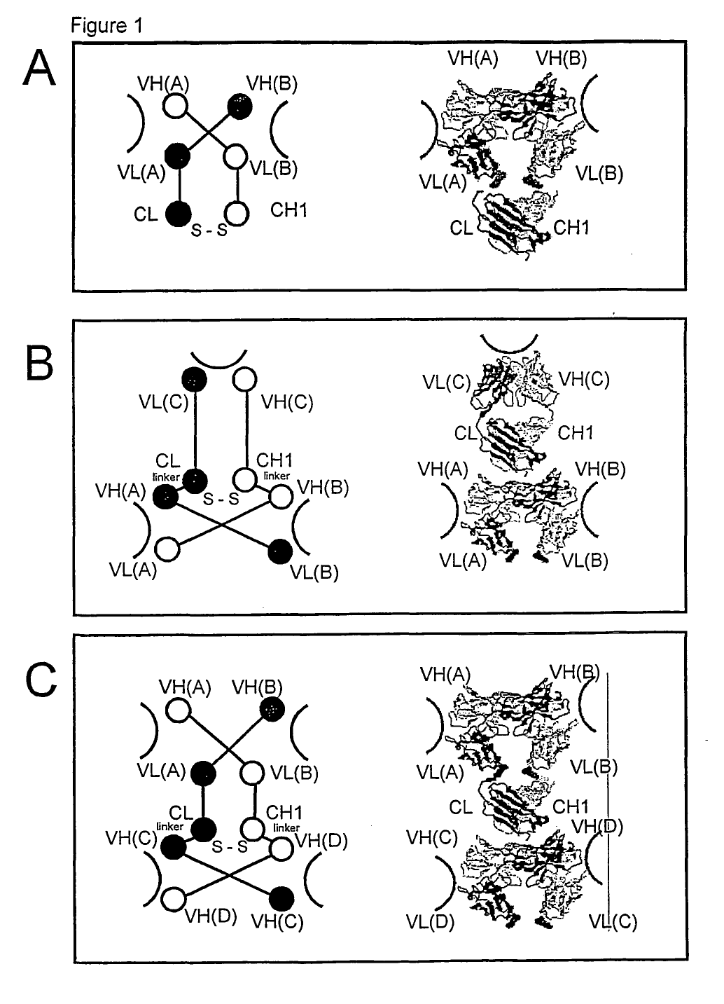

The arches indicate the antigen binding sites in the molecule.

FIG. 2: schematic representation of the gene structure after recombination of the DNA pieces encoding the desired protein domains.

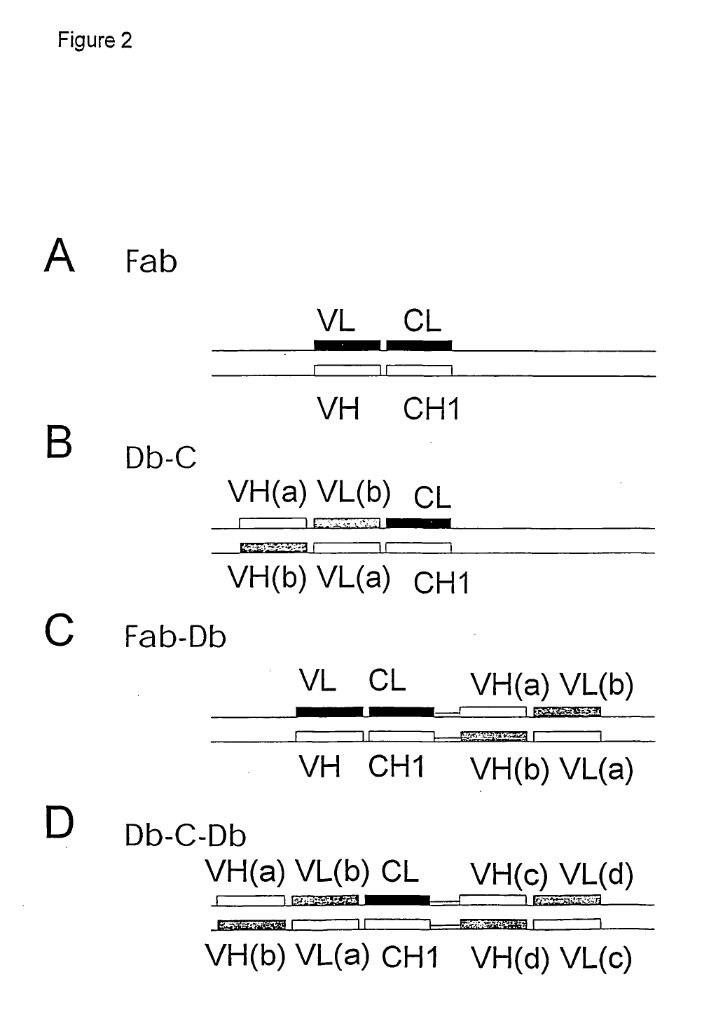

FIG. 3: An immunoblot analysis of antibody fragments-secreted in the medium after co-expression of isolated CL and CH1 domains fused to a signal sequence with each other or complete Fab chains. A) non-reducing SDS-PAGE gels (10%) of culture supernatant of HEK293T cells (co)transfected with the indicated IgH and L chain domains were blotted onto nitrocellulose membranes and probed with anti-murine IgG γ/κ antiserum (A and C) or anti-E-tag mAb (B; lane 1, L:H1 analogue without E-tag; lane 2, irrelevant E-tag-enlarged protein as a positive control). Closed arrowheads, detected molecules. Open arrowheads, presumed position of undetected products. H1, β-lactamase linker CH1/E-tag fusion protein. M, molecular mass markers. B) similar experiment combining only the isolated CL and CH1 constant domains. C) schematic representation of the mechanism of secretion of Fab-chains.

FIG. 4: An immunoblot analysis of the dimeric diabody-CL (Db-C) fusion protein probed with anti mouse IgG (gamma/kappa) serum, after a separation on a non-reducing and a reducing SDS-PAGE gel. For comparison, the Fab-fragment and the unfused diabody expressed in similar conditions are also shown on the non-reducing blot.

FIG. 5: An immunoblot analysis of a heterodimeric fusion protein formed by the expression of a first diabody chain fused to CL tagged with E-tag (Db1-CL-E), and a second diabody chain fused to CH1 tagged with HIS-tag (Db2-CH1-H) (A). Medium of transfected cells was analysed by non-reducing SDS-PAGE and probed with anti mouse IgG (gamma/kappa) (B), anti HIS-tag (C) and anti E-tag antibodies (D).

FIG. 6: An immunoblot analysis of a heterodimeric fusion protein formed by the expression of a VL-CL fused to a (GGGGS)₃ linker and to a first diabody chain (L-Db1), and a second chain comprising the VH-CH1 domains fused via the said linker to a second diabody chain extended with a HIS-tag (Fd-Db2-H) (A). Medium of transfected cells was analysed by non-reducing SDS-PAGE and probed with anti mouse IgG (gamma/kappa) (B) or anti HIS-tag (C) antibodies. The filled arrow indicates the heterodimeric fusion protein formed.

### DETAILED DESCRIPTION OF THE INVENTION

The invention relates to the nucleic acids encoding and methods for producing novel antibodies, comprising a heterodimeric fusion protein comprising two chains where the first chain comprises one or more variable domains of immunoglobulin in a VH-VL or VL-VH format coupled to a first heterodimerization domain and the second chain comprises one or more variable domains of immunoglobulin in a similar format as said first chain and coupled to a second heterodimerization domain interacting specifically with the first heterodimerization domain, and where at least two domains of the said first chain have intrinsic affinity to two domains of the said second chain.

The invention relates more specifically to a method for creating a fusion protein by heterodimerizing one or more bispecific diabodies. Most specifically, the heterodimerizing fusion partners are the CL and CH1 constant domains found in a Fab molecule.

Diabodies are formed by dimerizing scFv molecules, where the intramolecular interaction of the variable domains (VH:VL) is replaced by an intermolecular interaction. The result is a dimer of two diabody chains (VHVL:VHVL) with a skewed fold, so that the antigen binding sites of the diabody are both directed towards the outside of the molecule. A diabodies structure can be induced by fusing variable domains of immunoglobulin molecules with a peptide linker, preferably too short to allow spanning from the C-terminus of the first domain to the N-terminus of the second domain. Diabodies comprise two chains. To obtain a monospecific bivalent diabody, a dimer of a single type of diabody chain should be formed: VH(A)VL(A):VH(A)VL(A).

Bispecific diabodies can also be made. In this case, two different chains are constructed: VH(A)VL(B):VH(B)VL(A). If we define VH(A)VL(B) as chain A and VH(B)VL(B) as chain B, after co-expressing said chains, a mixture of dimers comprising A:A, B:B and A:B formats will be formed. It is the merit of the present invention to control heterodimerization of said diabody chains by fusion to the CL and CH1 constant domains of an immunoglobulin chain. In particular, one species of diabody chain should be fused to the CL domain, and the other species of diabody chain should be fused to the CH1 domain. The CL and CH1 domains can and should preferably be chosen to be non-immunogenic or non-antigenic in respect to the host receiving the biologic compound in case of use for in-vivo diagnosis or therapy. As a result of this invention, a molecule with a higher molecular weight will be produced. This modification improves the serum persistence of the molecule and increases the amount of protein that is allowed to bind the target molecule.

Preferably, the CL and CH1 domains should contain enough information to allow the intermolecular disulfide bridge to be formed. When oxidized, this will improve the stability of the resulting heterodimeric fusion protein.

Due to quality control in the endoplasmatic reticulum, unpaired CH1 domains do not proceed along the secretion pathway unless they are paired with an appropriate CL domain. This quality control is exerted by the chaperone BiP (GRP78), which binds most strongly to the CH1 domain and retains it until it is replaced by the appropriate interaction partner. Said partner could be the CL domain alone, but for many antibodies the CL domain alone will not be able to displace BiP from CH1. In this case, interaction of the complete L chain with the complete Fd-fragment of the H-chain is needed to replace BiP. A diabody can substitute the function of the VL and VH interaction in replacing BiP from CH1. This is surprising, since the predicted molecular conformation of a diabody fused to the CL and CH1 domains is very different from the natural Fab conformation. The symmetry axis of the binding interface of the diabody chains coupled to the CL and CH1 constant domains in not even in the same plane as the symmetry axis of the binding interface of the VL:VH interaction, which is in the same plane as the symmetry axis of the binding interface between CL and CH1.

The diabody chain can be fused to CL or CH1 without any additional linker sequences inserted. The diabody chains can be fused to the N-termini of the constant domains. The preferred fusion site would then be behind the peptide region connecting the constant and the variable region in the Fab, often referred to as “the elbow” region. Other fusion sites are also possible but it can be predicted that the optimal fusion point will depend on the conformation of the chosen diabody chains and of the conformation of the chosen constant domains. It is recommended to screen for the optimal fusion point by making fusions at different points, all or not including insertion of additional amino acids to serve as a linker region to avoid sterical constraints in the fusion protein. These additional amino acid linker can contain any sequence preferred, but again can be optimized according to the structure of the chosen fusion partners. Optimization of the chosen fusion point or of the interconnecting linker sequence can be done by using predictive algorithms as they are known in the art, or by an experimental approach, where different possible conformations are compared.

The diabody chains can also be fused to the C-terminus of the constant domains. In this case it can be predicted that insertion of additional amino acids to serve as a linker sequence between the constant domains and the diabody chain will improve the expression and stability of the molecule. Linker sequences are described in the art and can also be predicted by a person skilled in the art. Preferably, the linker sequence will be sufficiently flexible. Also preferably, a linker sequence should be chosen with low antigenicity. Natural occurring flexible linker sequences can be found in the Brooklyn Protein database of 3D structures (http://pdb-browsers.ebi.ac.uk//index.shtml) or in a sequence database such as the one hosted by the National Centre for Biotechnology Information NCBI (http://www.ncbi.nlm.nih.gov/).

Two diabodies can also be fused to the constant domains. In this case, the preferred method comprises fusing one diabody to the N-terminus of the constant domains and one diabody to the C-terminus of the constant domain. It is advisable to first optimize a structure containing only one diabody, C- or N-terminally fused. After optimization of each structure, a combination of both can be made. This will result in a heterodimeric molecule of the formula Db1-CL-Db2:Db1′-CH1-Db2′, whereby the diabodies are formed by interaction of two diabody chains (Db in formula). Preferably, but not limiting, the diabody should be of the formula VH(A)-VL(B):VH(B)-VL(A), where A and B denote a different antigen specificity.

It is thus clear that, when producing a heterodimeric diabody, two bivalent monospecific and a bispecific molecule can be formed. By combining different diabodies, it is possible to create antibody derivatives with two, three or four different specificities (bispecific, trispecific or tetraspecific). It is also possible to create a bispecific antibody where each specificity is formed by a bivalent binding, thus increasing the avidity of binding. Also a trispecific antibody can be formed where one specificity is formed by a bivalent binding.

The term “antibodies” means complete antibody molecules, antibody fragments or antibody derivatives. With antibody derivatives we mean all proteins comprising some part of an immunoglobulin protein, either fused in an non-natural way or not fused to other immunoglobulin parts or to other proteins or substances.

The term ‘intrinsic affinity’ refers to the ability of domains within the same protein to interact with each other. The said interaction can be weak. The said protein can be a fusion protein.

The term ‘fusion protein’ is used to indicate a single polypeptide or a combination of polypeptide chains where at least one polypeptide chain comprises different domains or peptide sequences derived from different sources.

When a diabody is fused through its N-terminus to the CL:CH1 domain pair, it is clear that now the new fusion protein is a heterodimerizing entity by itself. This heterodimerizing entity can be further coupled to other protein domains, complete proteins, subunits or peptides.

All genes for said fusion proteins should be assembled to a functional reading frame, either by assembling the encoding DNA to one open reading frame, or by the appropriate insertion of introns into the coding sequence. The genes encoding the fusion proteins should be operationally linked to functional translation and transcription signals for the host cell of choice, and linked to said expression signals placed on a DNA vector that can replicate in the host cell of choice, or can integrate in the genomic structure of the host cell of choice.

Heterologous host cells for the production of recombinant proteins are known in the art, and can for example, but not limiting, be a bacterium, a yeast or fungi cell, a plant cell, or any eukaryotic cell, e.g. insect cells and mammalian cells. Complete plant- or animal organisms comprising cells that produce the recombinant product are also known in the art. The product can also be produced by transgenic animals, e.g. in milk or in eggs, or in transgenic plants, e.g. in leaves or in seeds.

After production the recombinant heterodimeric fusion protein can be recovered by clearing and/or purification on the basis of its charge, hydrophobicity and molecular weight, and/or by affinity interaction with a ligand known to bind the heterodimeric fusion protein. Such a ligand could by example, but not limiting to, be one of the antigens recognized by one of the diabodies, or a specific tag sequence added to the fusion protein.

It should be clear for a person skilled in the art that the heterodimeric fusion proteins, and in particular de diabodies, of the present invention can be used in an identical or very similar manner as is described with regard to the usage of multispecific binding proteins in U.S. Pat. No. 5,837,242 to Holliger et al. and with regard to the usage multipurpose antibody derivatives in WO 99/37791 to Schoonjans et al. Both relevant parts in the descriptions of the latter patent applications are thus incorporated herein by reference.

It should also be clear that the heterodimeric fusion proteins of the present invention can also be used to allow transfection of specific target cells with, for example, retroviruses via using diabodies of the present invention that guide said retroviruses to said target cells by binding to a receptor specifically expressed by said target cells.

More specifically, the present invention relates to the usage of the heterodimeric fusion proteins of the present invention in diagnosis and therapy of diseases such as cancer, infectious diseases, autoimmune diseases, thrombosis etc. In this regard, the present invention thus also relates to pharmaceutical compositions comprising an immunotherapeutically effective amount of one or more heterodimeric fusion proteins according to this invention, or derivatized form(s) thereof and, preferably, a pharmaceutically acceptable carrier. By “immunotherapeutically effective amount” is meant an amount capable of lessening the spread, severity or immunocompromising effects of diseases as indicated above. By “pharmaceutically acceptable carrier” is meant a carrier that does not cause an allergic reaction or other untoward effect in patients to whom it is administered. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the heterodimeric fusion proteins. The compositions of this invention may be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application. The preferred compositions are in the form of injectable or infusible solutions. The preferred pharmaceutical compositions of this invention are similar to those used for passive immunization of humans with other antibodies. The preferred mode of administration is parenteral. It will be apparent to those of skill in the art that the immunotherapeutically effective amount of heterodimeric fusion proteins of this invention will depend, inter alia, upon the administration schedule, the unit dose of heterodimeric fusion proteins administered, whether the heterodimeric fusion proteins is administered in combination with other therapeutic agents, the immune status and health of the patient, and the therapeutic activity of the particular heterodimeric fusion protein administered. In monotherapy for treatment of the above-indicated diseases, immunotherapeutically effective amounts per unit dose of a heterodimeric fusion protein of the present invention range from about 0.1 to 10 mg/kg patient weight, preferably 2 mg/kg patient weight. Unit doses should be administered from twice each day to once every two weeks until a therapeutic effect is observed, preferably once every two weeks. The therapeutic effect may be measured by a variety of methods, including infectious agent load, lymphocyte counts and clinical signs and symptoms. It will be recognized, however, that lower or higher dosages and other administration schedules may be employed.

In another embodiment of the present invention relating to diagnosis, sample molecules may be allowed to bind or adhere to a solid support and the molecules so immobilized may be recognized by formation of reaction complexes with the heterodimeric fusion proteins of the present invention, through subsequent assay steps to detect reaction complexes. In a further embodiment, the heterodimeric fusion proteins of the present invention are bound to a solid phase support, for instance as the first component of a “sandwich-type” assay for molecules reactive with the heterodimeric fusion proteins of the present invention, wherein the second immunological binding partner may be a polyclonal or a monoclonal antibody, or a mixture thereof, including without limitation a heterodimeric fusion proteins of the present invention.

It is clear that the present invention further relates to any diagnositic method known in the art based on the usage of antibodies. In this regard, the invention also provides convenient test kit formats for practicing the foregoing methods.

In order that this invention may be better understood, the following examples are set forth. These examples are for purposes of illustration only, and are not to be construed as limiting the scope of the invention in any manner.

### EXAMPLES

### Example 1

### Release of CH1 from the ER chaperone BiP requires interaction of complete Fab chains

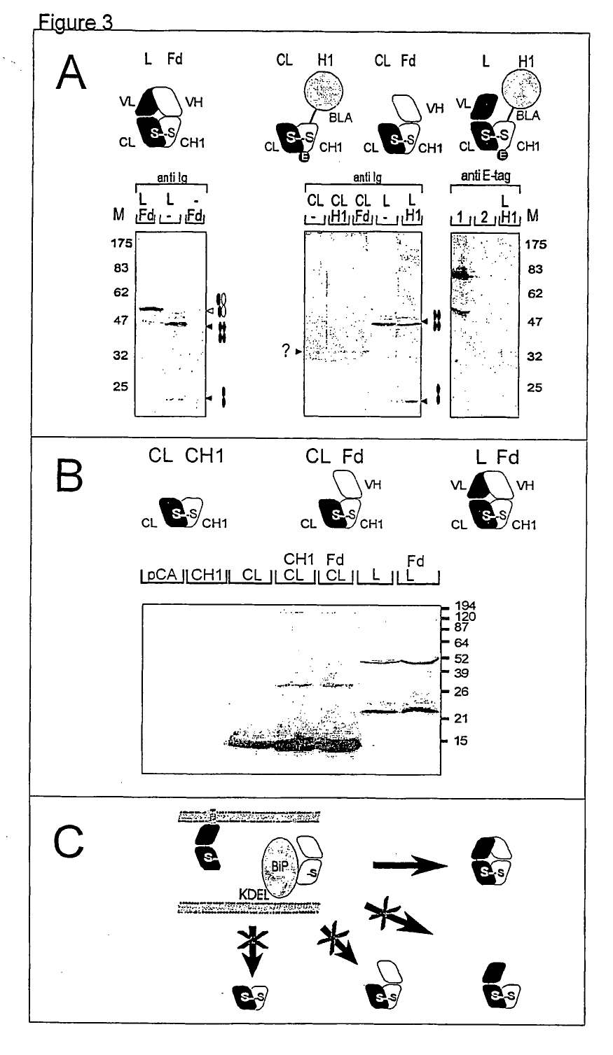

To assess the eukaryotic secretion of homo- and heterodimers from individual domains of Ab L and Fd chains, HEK293T cells were transiently (co)transfected with pCAGGS expression vectors containing as an insert either the CL or the CH1 domain. These domains are derived from mouse Ab E6 (IgG2b,κ), specific for hPLAP. In order to distinguish between CH1 and CL monomers and dimers, and CH1:CL heterodimers by molecular mass, the CH1 domain was N-terminally extended with 30-kDa P-lactamase, a bacterial protein which is efficiently secreted in mammalian cells. The CH1 domain was further modified with a C-terminal E-tag sequence to allow highly sensitive immunodetection of the product. As shown in FIG. 3A, co-expression of the lactamase linker CH1/E-tag fusion protein with the CL domain did not lead to a detectable heterodimeric product in the culture medium. To assess whether the presence of either the VH or the VL domains is required for progression of these Ab derivatives through the endoplasmic reticulum, the CL and CH1 domains were co-expressed with, their corresponding extended counterparts, namely the complete Fd chain and the native L chain, respectively. Also here, no secreted heterodimers, either CL:Fd or L:H1, could be detected, even with highly sensitive anti-E-tag detection (FIG. 3A). Only L monomers and L:L homodimers were detected in culture fluids of L gene-(co)transfected HEK293T cells. However, co-expression of CL and CH1, both enlarged with their corresponding variable domains (L and Fd chains) generated efficient expression of L:Fd heterodimers, only a slight fraction of L:L homodimers being visible as a faint band at 47 kDa (FIG. 3A). The Fd chain on its own was never detectable, neither as a monomer nor as a homodimer. Thus the Fd chain can only be secreted in the form of a heterodimer with the L chain, while the L chain preferentially forms heterodimers with the Fd chain upon co-expression.

This was confirmed in a second experiment where the CL and CH1 domains were fused directly to a signal sequence and transfected to HEK293T cells, either alone (CL and CH1) or in combination with each other (CL:CH1), or in combination with their opposite complete Fab-chain (CL:Fd and L:CH1). Induced protein was detected with anti mouse kappa light chain serum and only showed the CL monomer and disulfide stabilized CL:CL dimer (non-reducing SDS-PAGE). There was no detection of heterodimeric protein unless the complete L chains were co-transfected with the complete Fd chains to form a Fab fragment (FIG. 3B).

These results are in agreement with data obtained by other groups in studying BiP (Lee et al, 1999). Our data and these literature data favour the hypothesis that in order to displace BiP from CH1 a displacement energy should be developed that is equal or greater to the binding energy of CL to CH1. This may of course vary from antibody to antibody, since there is variability in the sequence of CH1. Also, in cells containing less BiP or when the expression of BiP is impaired, one can expect an exception to this finding. In the resulting working hypothesis BiP is not displaced by the interaction of CL with CH1 alone, but need the additional interaction energy delivered by the VL and VH interaction to allow the Fab chain to be secreted (FIG. 3C).

### Example 2

### Expression of a Diabody-Constant Domain Fusion Leads to a Disulfide-Stabilized Dimer

Since in the predicted structure of the diabody-constant domain fusion proteins the symmetry axis of the diabody is predicted to be perpendicular to the symmetry axis of the constant domains and not parallel to it as in the case of a VL:VH interaction, it was not obvious that this structure would be able to form.

A diabody was created by recombinant DNA methods and operationally fused to a promoter and a signal sequence functional in a mammalian cell. This diabody gene was then, also by genetic engineering, coupled to the constant domain of the E6 anti hPLAP murine IgG2b,κ antibody. This coupling was done in such a way that the complete coding sequence of the constant domain was present. The fusion point was chosen to be at the end of the variable domain and the beginning of the “elbow region”. The elbow region is here defined as the amino-acid sequence connecting the variable and the constant domains. These elbow regions can easily be identified from structural data present in several public databases, containing data regarding the primary structure of immunoglobulin domains, or in the Brookhaven Protein Database for structural data. When working with antibodies not listed in any of those databases, this region can easily be determined by homology with known structures.

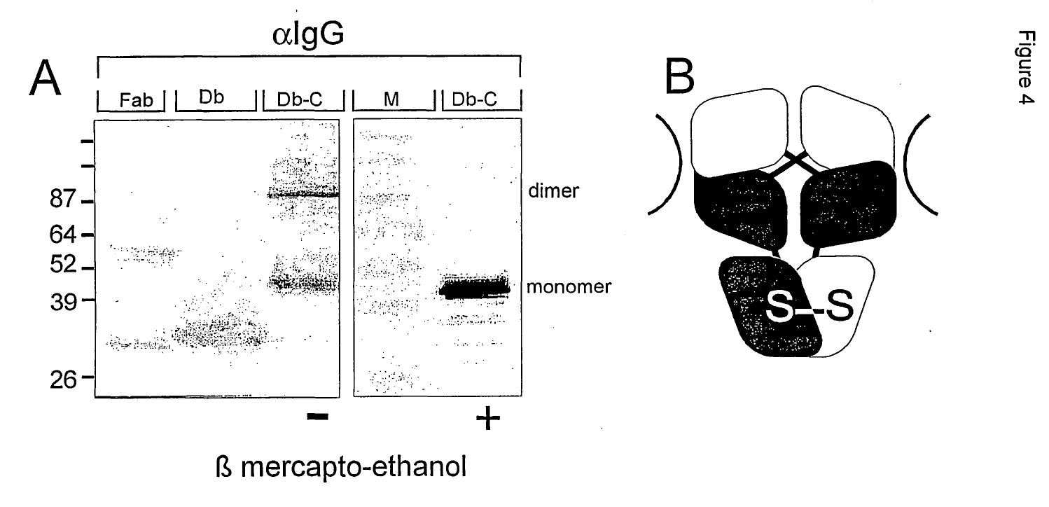

In FIG. 4 an example is shown where a diabody chain id fused to a CL constant domain. Expression of the diabody-constant domain fusion clearly showed the presence of a disulfide stabilized dimer, which was dissociated upon treatment with a reducing agent such as β-mercapto-ethanol, known to break disulfide bonds in proteins. The presence of the disulfide bridge indicates a close proximity of the constant domains in both chains, which is a clear indication that the predicted fusion protein is formed.

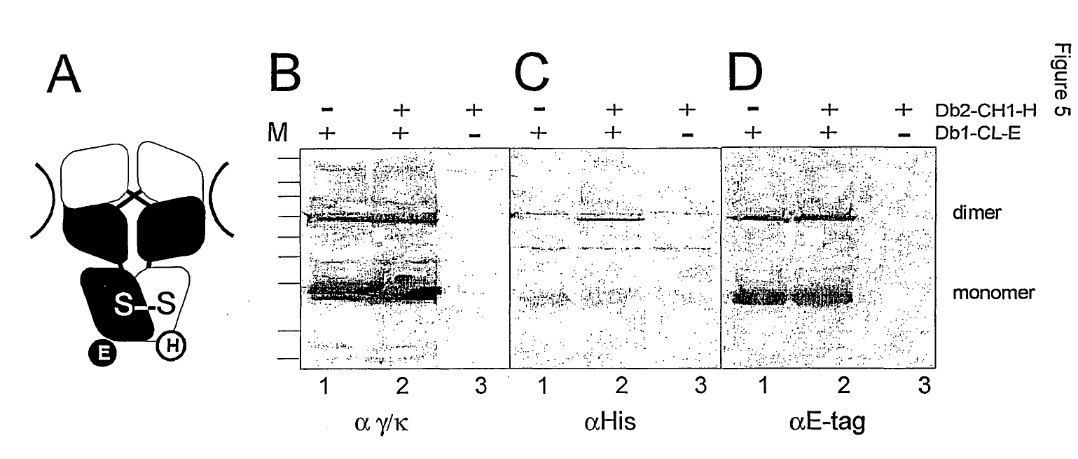

In FIG. 5 an example is shown where a firs diabody chain is fused to the CL constant domain, C-terminally extended with an E-tag peptide. A second diabody chain is fused to the CH1 constant domain and C-terminally extended with a HIS-tag peptide. These genes were transfected either alone or in combination and the medium of the cells was analysed for secreted antibody fragments by probing with anti IgG (gamma/kappa), anti-HIS tag or anti E-tag serum. The immunoblots show that the diabody-CL fusion protein can be secreted when transfected alone, and that a disulfide-stabilized dimer is present. As expected, the diabody chain-CH1 fusion protein is not detected when transfected alone. When co-transfected with the diabody chain-CL fusion protein however, a disulfide stabilized dimer is formed that can be detected with anti-HIS-tag and with anti-E-tag monoclonal antibodies, indicating the presence of both chains in the dimer.

### Example 3

### Expression of a Heterodimeric Fusion Protein Comprising Diabody Chains Fused to the C-terminus of the CL and CH1 Domains

From the structural data it can be predicted that fusion of the N-termini of a diabody dimer to the C-termini of a Fab-fragment benefits from the insertion of a peptide linker. In the example shown, a linker sequence of the formula (GGGGS)₃ is used, where G=glycin and S=serine. It will be clear for a person skilled in the art that other suitable linker sequences can be found without the involvement of an inventive step. In order to allow the formation of a C-terminal disulfide bond between the CL and the CH1 domain, the sequence chosen for these domains contains the appropriate C-terminal cystein amino acid, and the fusion point of the linker sequence should be chosen accordingly.

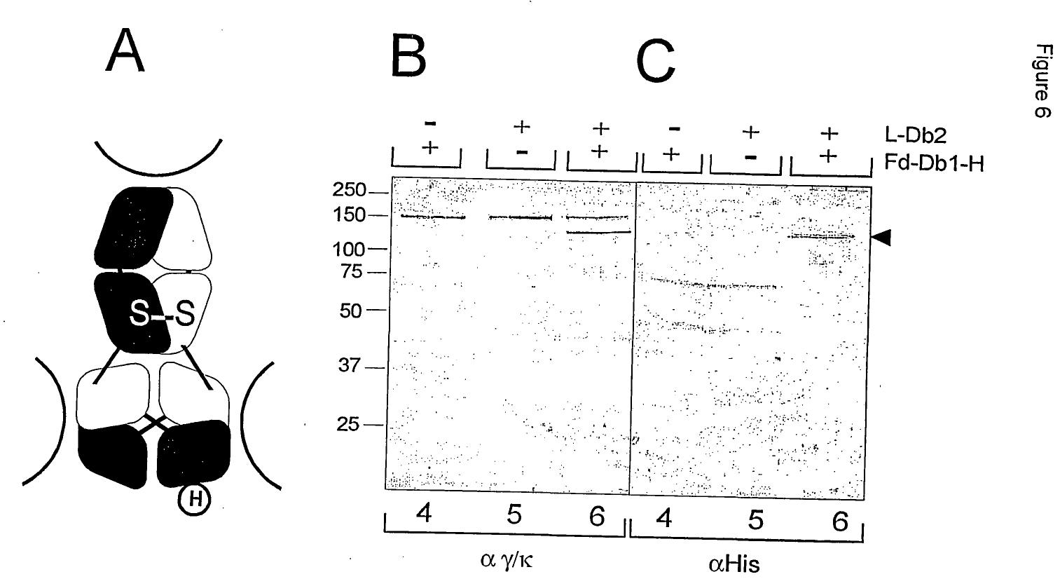

In the example shown in FIG. 6, the CL and CH1 domains are extended with their appropriate VL and VH domains, to ensure a proper secretion from the cells. In this case, the Fd-diabody chain fusion protein was C-terminally tagged with a HIS-tag. Both the L-diabody chain and the Fd-diabody chain-HIS tag were transfected either alone or in combination with each other. The immunoblot shown in FIG. 6B shows a larger non-specific band, and a single protein upon co-transfection, that also reacts with anti-HIS tag antibody. In this case, the L-diabody chain was very weakly expressed. Considering our data that prove that Fd-chains or fusion proteins containing Fd chains are not secreted from the cell unless paired with a L-chain or L-chain fusion, it can be concluded that the fusion protein produced upon co-transfection of both fusion chains is the heterodimeric Fab-bispecific diabody.

### Example 4

### Expression of a Heterodimeric Fusion Protein Comprising Diabody Chains Fused to the N-terminus and to the C-terminus of the CL and the CH1 Domains

A preferred method is to start from a pair of genes encoding diabody-constant domain (CL and CH1) fusions where the diabody is N-terminally fused and the constant domain is the C-terminal domain in the fusion chain. A second pair of genes then encodes fusion genes where the constant domains CL and CH1 are the N-terminal domains and the diabody chains are fused to the C-terminus. Both pairs of genes are adapted to optimal expression in the chosen host. Co-expression of each pair of genes reveals the relative expression level obtainable. By using standard DNA manipulation techniques, including PCR (polymerase chain reaction) approaches, changes can be made to either the expression signals or to the protein structure. It may be necessary to modify the fusion point or the linker sequences to obtain a better production of heterodimeric fusion protein. This may be an iterative process that ends when the result is satisfactory.

By using standard DNA cloning techniques a final pair of fusion genes is created encoding a diabody chain—constant domain (CL or CH1)—diabody chain fusion chain. This can be done by using restriction endonucleases and ligases or by splice overlap extension PCR. The final product is preferentially checked for integrity preferentially by DNA sequence analysis. Both recombinant fusion genes are then checked for expression of the final fusion protein by co-expressing the both fusion genes obtained in the chosen host cell.

### Example 5

### Functional Binding of Heterodimeric Fusion Proteins

If antigen bound by the antigen binding sites comprised by the fusion protein is available in sufficient amount, it can be used to coat on a solid support such as an ELISA plate. The fusion protein containing one or multiple diabody molecules can then be enriched, purified, or used directly to bind the coated antigen. Bound diabody containing fusion proteins can then be detected by species-specific anti-immunoglobulin serum that was tested and approved for binding to variable domains or CL and CH1 domains. Fab-specific serum or antibody usually fulfils these requirements. If this serum is not conjugated to an enzyme allowing detection, a second serum or monoclonal antibody interacting with the first serum or monoclonal antibody, where the second serum or monoclonal antibody is conjugated with an enzyme that allows detection. Detection systems and signal development is well known in the art.

As an alternative, a second antigen can be used to interact with the bound diabody containing fusion protein, after which the second antibody is detected with serum or a monoclonal antibody as described.

If multiple specificities are present in the diabody containing fusion proteins, as much combinations of antigen as possible are assayed.

These binding assays will confirm the functionality of the diabody containing fusion protein and if appropriate titration curves are performed by diluting the diabody containing fusion protein or by competition with uncoated primary antigen an estimate to the affinity of the antigen-antibody derivative can be made.

To refine these estimates, techniques based on surface plasma resonance are known in the art and allow kinetic analysis of the binding parameters.

In order to check for functional binding on cells expressing the antigen, fluorescence-based flow cytometry can be used as described in Schoonjans et al., 2000.

In order to check on other functions aimed at during the creation of the bispecific or multispecific recombinant antibody derivative, a specific assay is created. If e.g. one of the functions aimed for is the activation of T-cells, a T-cell proliferation assay or a T-cell cytotoxicity assay can be set up as described in Schoonjans et al., 2000.

The development of a binding assay for the recombinant diabody containing fusion protein id preferred not only to generate data on binding characteristics, but also to assay for functional protein during expression, downstream processing, and purification procedures.

The development of a functional assay for the created molecule is preferred in order to generate data on the specific activity of the novel protein.

### Example 6

### Testing Therapeutic Use

Molecules with a potential therapeutic use are tested in a relevant animal model. For model development one can make use of mouse genetics to select an appropriate mouse strain. Appropriate settings are defined by experimental conditions where a maximal read-out is obtained from the effect of the recombinant antibody. Mice are then treated with dilutions of the recombinant antibody to determine the minimal effective dose, the minimal frequency of administration and the maximal effect of the new therapeutic compound. If relevant for the use of the recombinant antibody, the fusion protein can be labelled e.g. by coupling with gamma emitting radioactive salts, after which the biodistribution of the compound to different organs can be compared to the binding of the target organ or target cells. In a similar way, the clearance rate of the fusion protein is determined.

Bispecific or multispecific fusion proteins might also be designed to clear antigen (including but not limited to haptens, allergens, proteins, viruses, bacteria and parasites) from the blood stream by crosslinking the target to red blood cell receptors or other receptors responsible for antigen clearing. In this case the antigen is injected into the animal, followed by an injection of recombinant bispecific antibody. The remaining antigen concentration in the blood serum is then determined in function of time of treatment start or dose of the recombinant bispecific antibody used.

### REFERENCES

Alt, M., Muller, R. and Kontermann, R. E.: Novel tetravalent and bispecific IgG-like antibody molecules combining single-chain diabodies with the immunoglobulin gamma1 Fc or CH3 region. FEBS Left 454 (1999) 904.

Brennan, M., Davison, P. F. and Paulus, H.: Preparation of bispecific antibodies by chemical recombination of monoclonal immunoglobulin G1 fragments. Science 229 (1985) 81-83.

Coloma, M. J. and Morrison, S. L.: Design and production of novel tetravalent bispecific antibodies. Nat. Biotechnol. 15 (1997) 159-163.

Glennie, M. J., McBride, H. M., Worth, A. T. and Stevenson, G. T.: Preparation and performance of bispecific F(ab′ gamma)₂ antibody containing thioether-linked Fab′ gamma fragments. J. Immunol. 139 (1987) 2367-2375.

Hayden, M. S., Linsley, P. S., Gayle, M. A., Bajorath, J., Brady, W. A., Norris, N. A., Fell, H. P., Ledbetter, J. A. and Gilliland, L. K.: Single-chain mono- and bispecific antibody derivatives with novel biological properties and antitumour activity from a COS cell transient expression system. Ther. Immunol. 1 (1994) 3-15.

Holliger, P., Prospero, T. and Winter, G.: “Diabodies”: small bivalent and bispecific antibody fragments. Proc. Natl. Acad. Sci. U.S.A. 90 (1993) 6444-6448.

Hu, S., Shively, L., Raubitschek, A., Sherman, M., Williams, L. E., Wong, J. Y., Shively, J. E. and Wu, A. M.: Minibody: A novel engineered anti-carcinoembryonic antigen antibody fragment (single-chain Fv-CH3) which exhibits rapid, high-level targeting of xenografts. Cancer Res 56 (1996) 3055-61.

Karpovsky, B., Titus, J. A., Stephany, D. A. and Segal, D. M.: Production of target-specific effector cells using hetero-cross-linked aggregates containing anti-target cell and anti-Fc gamma receptor antibodies. J Exp Med 160 (1984) 1686-701.

Kipriyanov, S. M., Dubel, S., Breitling, F., Kontermann, R. E. and Little, M.: Recombinant single-chain Fv fragments carrying C-terminal cysteine residues: production of bivalent and biotinylated miniantibodies. Mol Immunol 31 (1994) 1047-58.

Kipriyanov, S. M., Moldenhauer, G., Schuhmacher, J., Cochlovius, B., Von der Lieth, C. W., Matys, E. R. and Little, M.: Bispecific tandem diabody for tumor therapy with improved antigen binding and pharmacokinetics [In Process Citation]. J Mol Biol 293 (1999) 41-56.

Kostelny, S. A., Cole, M. S. and Tso, J. Y.: Formation of a bispecific antibody by the use of leucine zippers. J. Immunol. 148 (1992) 1547-1553.

Mack, M., Riethmuller, G. and Kufer, P.: A small bispecific antibody construct expressed as a functional single-chain molecule with high tumor cell cytotoxicity. Proc. Natl. Acad. Sci. U.S.A. 92 (1995) 7021-7025.

McGregor, D. P., Molloy, P. E., Cunningham, C. and Harris, W. J.: Spontaneous assembly of bivalent single chain antibody fragments in Escherichia coli. Mol Immunol 31 (1994) 219-26.

Milstein, C. and Cuello, A. C.: Hybrid hybridomas and their use in immunohistochemistry. Nature 305 (1983) 537-540.

Muller, K. M., Arndt, K. M. and Pluckthun, A.: A dimeric bispecific miniantibody combines two specificities with avidity. FEBS Left 432 (1998a) 45-9.

Muller, K. M., Arndt, K. M., Strittmatter, W. and Pluckthun, A.: The first constant domain (C(H)1 and C(L)) of an antibody used as heterodimerization domain for bispecific miniantibodies. FEBS Lett 422 (1998b) 259-64.

Pack, P. and Pluckthun, A.: Miniantibodies: use of amphipathic helices to produce functional, flexibly linked dimeric FV fragments with high avidity in Escherichia coli. Biochemistry 31 (1992) 1579-1584.

Ridgway, J. B., Presta, L. G. and Carter, P.: ‘Knobs-into-holes’ engineering of antibody CH3 domains for heavy chain heterodimerization. Protein Eng. 9 (1996) 617-621.

Schoonjans, R., Willems, A., Schoonooghe, S., Fiers, W., Grooten, J. and Mertens, N.: Fab chains as an efficient heterodimerization scaffold for the production of recombinant bispecific and trispecific antibody derivatives. J Immunol 165 (2000) 7050-7.

Zhu, Z., Presta, L. G., Zapata, G. and Carter, P.: Remodelling domain interfaces to enhance heterodimer formation. Protein Sci 6 (1997) 781-8.

1. A heterodimeric fusion protein, comprising two chains where the first chain comprises domains that all have intrinsic affinity for a corresponding domain in the second chain, and where the heterodimerization of both chains is controlled by incorporating chosen domains that are known to constitute a preferred heterodimer.

2. A heterodimeric fusion protein as in claim 1, where the said chosen domains are selected from the CL and CH1 constant immunoglobulin domains.

3. A heterodimeric fusion protein as in claim 1 and/or 2, where the first and the second chain comprises one or more diabody chains, and form one or more functional diabodies after heterodimerization.

4. A heterodimeric fusion protein as in claim 3, where the first chain and the second chain form two distinct bispecific diabodies within the fusion protein by heterodimerization resulting in a tetraspecifc antibody derivative.

5. A heterodimeric fusion protein as in claim 3, where the first chain and the second chain form two identical bispecific diabodies within the fusion protein by heterodimerization, resulting in a bispecific antibody derivative where each said specificity is formed by a bivalent binding.

6. A heterodimeric fusion protein as in claim 3, where the first chain and the second chain form diabodies within the fusion protein by heterodimerization resulting in a recombinant antibody derivative with one bivalent binding specificity, and two other specificities.

7. A heterodimeric fusion protein as in claim 1-6, that is further extended at one or more of its N-terminal or C-terminal ends with independent folding additional protein domains, protein subunits, complete proteins, protein fragments or peptides.

8. Heterodimeric fusion proteins as in claim 1-7 for use as a medicament.

9. Use of heterodimeric fusion proteins as in claim 1-9 for the preparation of a medicament to prevent and/or treat cancer, infectious diseases, autoimmune diseases and thrombosis.

10. Use of heterodimeric fusion proteins as in claim 1-7 for use in a diagnositic kit to diagnose cancer, infectious diseases, autoimmune diseases and thrombosis.

11. One or more DNA constructs encoding the domains needed to constitute the heterodimeric fusion proteins of claim 1-7, comprising suitable transcription and translation regulatory sequences operably linked to sequences encoding the said heterodimeric fusion proteins.

12. Method for producing heterodimeric fusion proteins as claimed in claims 1-7, comprising expression of one or more DNA constructs as claimed in claim 11 in heterologous expression host cells.

13. Method as claimed in claim 12, wherein the host cells are E. coli cells, other bacterial cells, such as Bacillus spp., Lactobacillus spp. or Lactococcus spp.; actinomycetes; yeasts; filamentous fungi; mammalian cells such as COS-1 cells, HEK cells, myeloma cells or CHO cells, insect cells, transgenic animals or plants.
